(19) United States
(12) Patent Application Publication
(10) Pub. No.: US 2010/0048758 A1
(43) Pub. Date: Feb. 25, 2010

(54) LUBRICIOUS COATING COMPOSITION FOR DEVICES

(75) Inventors: Hancun Chen, Maple Grove, MN (US); James G. Hansen, Minneapolis, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., MAPLE GROVE, MN (US)

(21) Appl. No.: 12/196,771

(22) Filed: Aug. 22, 2008

(51) Int. Cl. A61K 8/72; B05D 3/00

(52) U.S. Cl. 523/105; 427/2.1

(57) ABSTRACT

Alternative coating compositions and/or methods of coating a medical device. Some examples include coating compositions comprising a polyester urethane and a polyether in a substantially aqueous carrier. The coating composition may be provided as a one or two part system and may be applied by a variety of coating methods, in some cases without a requirement for special equipment or undue industrial hygiene concerns. Additionally, methods of coating medical devices with substantially aqueous coating compositions and the resulting coated medical devices. The composition may be applied to all or part of a medical device by spraying, dipping, brushing, extruding, or the like. After the coating composition has been applied, the substantially aqueous carrier, including any attendant cosolvents or other volatile species, may be removed, typically by heating with optional enhanced air flow although simple evaporation at ambient conditions may also be used.

### TECHNICAL FIELD

This invention relates generally to polymeric hydrophilic coating compositions for application to medical devices, methods of applying the coating compositions, and to articles coated therewith.

### BACKGROUND

There is an increasing desire in the medical arts to provide a variety of devices with increased lubricity relative to the materials commonly employed in their manufacture. This is particularly desirable when the devices are to be implanted or inserted into the body. Such medical devices may include guidewires, catheters such as guide catheters or catheters that are utilized to deliver a stent, a stent-graft, a vena cava filter, balloon catheters, and certain expandable medical devices such as basket filters. As minimally invasive surgical techniques have improved, it has become increasingly common to insert and retrieve medical devices through catheters and the like having considerable length. Accordingly, it is desirable to minimize friction between the catheters that carry such devices and the devices themselves as well as with tissue with which they may come in contact. In the past, the industry has employed various hydrophobic oils and coatings such as olive oil, silicone, and the like as lubricants. These materials tend to be displaced in the aqueous environments in which they are used and generally to lack abrasion resistance. In some patients, silicone has been known to cause tissue reaction and irritation. Generally, the shedding of debris of any sort is to be avoided.

Hydrophilic coatings, particularly hydrogels, also have been employed to impart lubricity to a variety of medical devices. Hydrogels alone may migrate from the surfaces to which they are applied when they are exposed to an aqueous environment. Prior art hydrophilic coatings typically have relied upon a two step, two coating process, usually involving a primer coat of isocyanate or isocynate/prepolymer, which is applied from a solvent and dried, followed by a second solvent-borne coating containing a hydrophilic component such as polyvinyl pyrrolidone or polyethylene oxide. The two coatings, one superimposed on the other, are then baked to effect a cure. One step coating application methods have also been described. In addition to isocyanate based curing systems, other chemistries, such as free radical mechanisms involving acrylate moieties, have been proposed.

### SUMMARY OF THE INVENTION

There is still a need for biocompatible, relatively inert, lubricious coatings for medical devices which can be easily and safely applied by a variety of coating methods.

Accordingly, the present invention relates to some alternative coating compositions and/or methods of coating a medical device. One example embodiment relates to coating compositions comprising a polyester urethane and a polyether in a substantially aqueous carrier. The coating composition may be provided as a one or two part system and may be applied by a variety of coating methods, in some cases without a requirement for special equipment or undue industrial hygiene concerns.

Some embodiments relate to methods of coating medical devices with substantially aqueous coating compositions and the resulting coated medical devices. The composition may be applied to all or part of a medical device by spraying, dipping, brushing, extruding, or the like. After the coating composition has been applied, the substantially aqueous carrier, including any attendant cosolvents or other volatile species, may be removed, typically by heating with optional enhanced air flow although simple evaporation at ambient conditions may also be used.

The above summary of some embodiments is not intended to describe each disclosed embodiment or every implementation of the present invention. The Figures, and Detailed Description, which follow, more particularly exemplify these embodiments.

### BRIEF DESCRIPTION OF DRAWINGS

The invention may be more completely understood in consideration of the following detailed description of various embodiments of the invention in connection with the accompanying drawings, in which:

FIG. 1 is a partial cutaway, side view of an example coated medical device, a guidewire.

FIG. 2 is a transverse section of the guidewire along line 2-2.

FIG. 3 is a cross-section of an apparatus spray coating a medical device.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

The present inventors understand that there is a desire for substantive coatings, applied to various medical devices, which are both lubricious and biocompatible. Additionally, it may be desired that these coatings be easily applied to a variety of substrates, such as metals and polymers, without a need for special equipment or complex processes. The coatings may be abrasion resistant, free from significant shedding, and free of components which are potentially irritating to persons, particularly medical patients, who may come in contact with them. It may be highly desirable that the coatings significantly reduce friction between the medical device and other components of a delivery system, especially in an aqueous environment such as bodily fluids. The coating may provide these benefits as a thin, generally flexible coating which is adherent to the substrate.

Solvent-borne coatings, particularly those containing isocynates, have been used in the preparation of similar coatings; however residual isocynates and even residual solvents have been known to result in tissue damage and allergic reactions. This is of particular concern when the damage or reaction occurs in a patient whose health is already compromised. In addition, the use of solvents and isocyanates in a manufacturing process may create unnecessary risk for the workers involved. Avoiding contact and inhalation exposures can be quite expensive. Limiting solvent release through solvent recovery can be difficult.

Although urethane polymers resulting from in situ reaction between isocyanates and reactive monomers such as polyester polyols are known to be abrasion resistant, they typically are at least somewhat hydrophobic and may be somewhat variable in the mechanical properties achieved. This variability has been attributed to side reactions with ambient water vapor or other contaminants and the resulting premature chain termination.

Accordingly, it may be desirable to provide a base polymer in a preformed state such as an aqueous colloidal dispersion or emulsion. This may allow the utilization of relatively high molecular weight polymers, in some embodiments near or above their entanglement molecular weights, which are substantially free of unreacted species such as isocyanates or other comonomers, for example aziridines, reactive with active hydrogen bearing groups. Residual isocyanate groups may typically be consumed in slower reactions with the aqueous phase before use in these coatings. Such high molecular weight polymers are commercially available and include polyester urethanes as well as polyether polyester urethanes. The liquid carrier associated with commercially available aqueous polyurethane dispersions and emulsions, suitable for use in coatings may include lesser amounts of solvents such as isopropanol or other coalescing aids while still being considered aqueous. Upon removal of the liquid component(s), whether at ambient or elevated temperature, the dispersed polyurethane component generally fuses into a coherent film without the need for additional reactions to promote chain extension and/or crosslinking. In some embodiments, however, it may be desirable to employ a post-coating reaction to further ensure the integrity of the resulting film. Typically, a reaction mechanism such as a free radical reaction of pendant acrylate or other unsaturated groups may be utilized to separate, in time, the initial polymerization which results in the dispersion or emulsion and any subsequent curing step. In other embodiments a crosslinker may be added prior to coating.

In addition to the polyester urethane and/or polyether polyester urethane component, it is desirable to provide a hydrophilic component, for example a polyether, such as a polyethylene oxide. The hydrophilic component may be supplied as a solution, gel, dispersion, or emulsion, with a solution or gel used in some embodiments. The hydrophilic component may be provided in the form of a copolymer, for example, a polyether urethane. The polyether component may be linear, branched, or present as pendant side-chains. It may be a homopolymer, random copolymer, or a block copolymer.

It will be appreciated that the selection of an application technique for coating a medical device with the substantially aqueous coating composition will depend to an extent upon the device to be coated and whether it is desirable to coat the entire device or only a portion thereof. Accordingly, a variety of application techniques such as spraying, dipping, brushing, extruding, curtain coating, or piece-part curtain coating may be appropriate in a particular situation. The invention contemplates medical devices bearing the coating compositions from which the volatile components substantially have been removed as part of the coating process.

Once an application technique, or in some instances more than one technique, has been selected, the components of the coating may be applied simultaneously and/or sequentially. In certain embodiments, the polyester urethane component and the polyether component may be combined prior to application. If the various components are not mutually reactive, they may be mixed to form a one part system. In some embodiments, it may be desirable or simply convenient to apply the polyester urethane component and the polyether component separately. In such a coating method, it is generally desirable that the coatings be applied simultaneously to ensure good mixing of the two components; however, other factors such as improved adhesion to a substrate may dictate a sequential application. In some embodiments, sequential application, especially with the polyether component applied after the polyester urethane component, may result in a useful coating composition gradient within the final coating. It may be desirable to employ different carrier compositions, the volatile components of a coating composition, in the various coating compositions to be applied. In still other embodiments, a first coating component may be applied, followed by at least partial removal of the carrier, application of the second component, and final carrier removal. In some applications, it may be useful to alternate applications of first and second components of the coating composition in order to build a thicker final coating or a coating with a stratified structure. In yet other embodiments, it may be desirable to apply a base coat comprising a single coating composition component, for example as an anchoring layer, followed by simultaneous or premixed application of both coating composition components.

Aqueous coating systems are often desirable for this application in that they avoid toxicological problems associated with worker exposure to common solvents involved in the formulation and coating operations. In addition, they avoid costly and potentially dangerous issues associated with solvent recovery and solvent, or solvent vapor, fires. Aqueous coating systems may include lesser amounts of solvents such as isopropanol or other coalescing aids which do not unduly react with other species present.

Without wishing to be bound by theory, it is believed that the polyester urethane component of the coating composition and the polyether component of the coating composition typically will not be fully compatible and may tend to segregate into separate domains as the carrier is removed. Accordingly, the coated medical articles may exhibit a range of coating morphologies depending upon the particular components, carriers, and conditions employed. The coatings may be fully homogeneous, microphase separated, macrophase separated, or they may form interpenetrating networks or semi-interpenetrating networks. In addition, certain pairs of polyester urethanes and polyethers are thought to form complexes which aid in the retention of the polyether component. With these possible morphologies in mind, it may be possible to alter the fraction of the surface of the completed, coated medical device which is predominantly hydrophilic by altering the ratio of the polyester urethane component to the polyether component as well as by altering the coating conditions.

In some embodiments, the coating composition may be applied to a medical device using conventional spray technology. It is possible to mix the two components prior to spraying or at the time of spraying. One or more spray heads may be used. In some embodiments, it is convenient to use multiple spray heads arrayed about the article to be coated to ensure quick and complete coverage. In such configurations, it may be desirable to configure the spray heads such that one group of one or more spray heads applies one component of the coating composition while a second group of one or more spray heads applies the second component of the coating composition. In yet other embodiments, a third component, for example one or more agent reactive with one or more of the coating composition components, may be applied by a third group of one or more spray heads. In one part or two part application methods, the third component, if present, may be added to one or both of the coating composition components prior to the application of the aqueous coating. It will be appreciated that other application methods may be adapted to simultaneous or sequential application of coating composition components in one, two, three, or even more part coating operations in a manner analogous to the spray methods described above.

Carrier removal may be accomplished by conventional methods including, but not limited to, passing warm, dry air over the coated medical device. The carrier may be removed in the coating environment or may be removed in a separate oven. In addition to carrier removal, in some embodiments it may be desirable to subject the coated medical device to mild heating to alter the morphology of the coating or to promote interactions between the components of the coating.

In some embodiments, it may be desirable to moisten or humidify the coating to restore moisture content after the coating process, which usually includes significant removal of the volatile, substantially aqueous carrier. Although it may be possible to limit the removal of the volatile carrier components to achieve a moistened state, it may be easier, or even preferable, to fully dry the coating and then to moisten the dried coating to the desired degree. The restored moisture content is thought to impart an immediately lubricious character to the coating on the medical device, rendering it more suited to direct insertion into the body. The coating may be rehumidified and stored in a moistened state and/or it may be moistened shortly before use. In some embodiments, it is believed that one or more moistening/drying cycles may alter the surface morphology of the coating in a desirable manner.

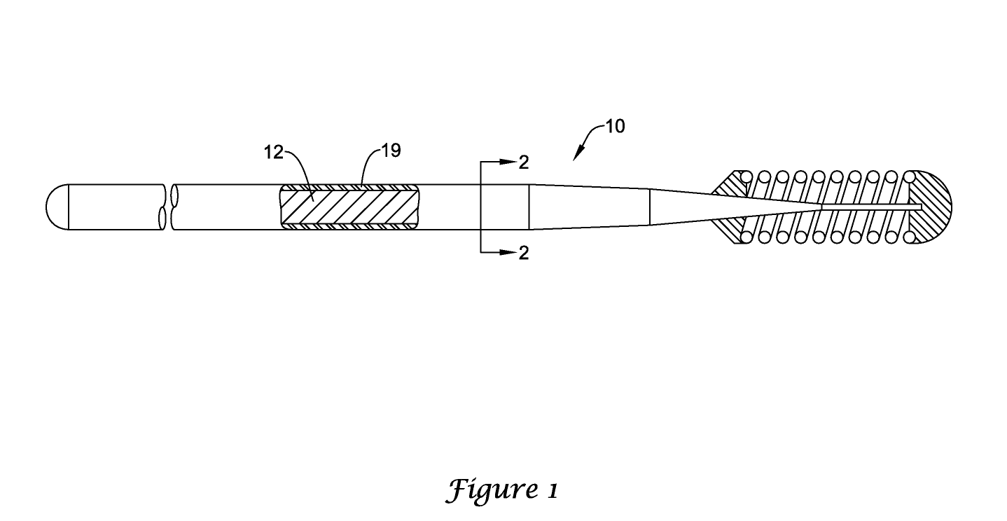

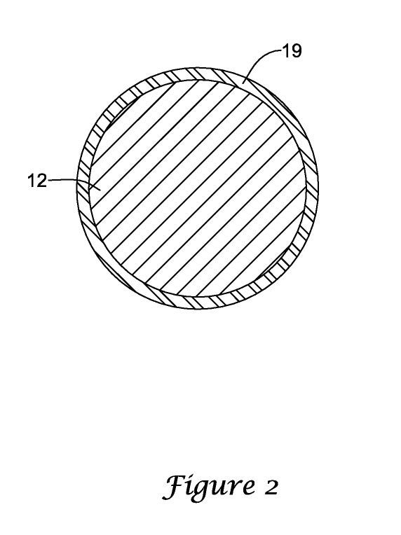

FIG. 1 is a partial cutaway, side view of a coated medical device, a coated guidewire 10. The guidewire 12 bears a coating 19 over substantially its entire length. The coating comprises two or more coating components, a polyester urethane and a polyether. The coatings may be fully homogeneous, microphase separated, macrophase separated, or they may form interpenetrating networks or semi-interpenetrating networks. In some embodiments, the coating is uniformly applied, while in other embodiments the coating thickness and/or coating composition may vary along the length of the example guidewire or around its circumference. FIG. 2 is a transverse cross-section of the example guide wire illustrating a substantially uniform coating thickness.

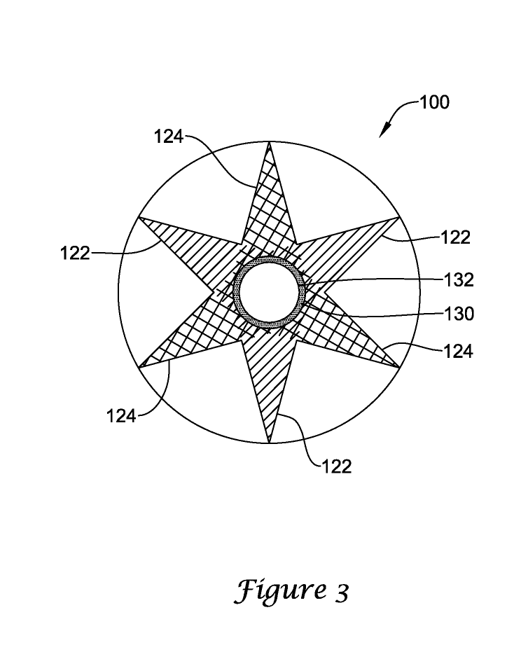

FIG. 3 is an illustrative cross-section of one spray coating apparatus 100 in which multiple spray patterns 122, 124 overlap and surround a medical device 132 to be coated. In the particular embodiment presented, the coating 130 is being applied as two separate components which combine on the medical device 132. For example, the aqueous polyester urethane may be applied as spray patterns 122 while the aqueous polyether component may be applied as spray patterns 124. In other embodiments, the aqueous polyester urethane component and the aqueous polyether component may be mixed prior to application and supplied to spray heads (not shown) which generate spray patterns 122 and 124 as a single component. In either event, spray patterns 122 and 124 may be applied simultaneously or sequentially. In preferred embodiments, the medical device 132 to be coated is rotated within the overlapping spray patterns 122, 124 and may be translated along the axis of the spray coating apparatus 100. Although the medical device 132 is depicted as generally centered within the spray pattern, it may be advantageous to deploy it in an off-centered position or even to vary its position within the spray patterns to achieve specific deposition patterns.

In some embodiments, the spraying operation may be accompanied by an axial flow of warm air to assist the removal of volatile components of the carrier. In other embodiments, impingement air may be supplied in a different portion of the coating apparatus. In yet other embodiments, the coating apparatus may include other components such as infrared lamps to heat the medical device before or after application of the coating composition or UV lamps to initiate a free radical cure of optional components of the coating.

In one embodiment, the polyester urethane of the coating composition is supplied as a colloidal dispersion or emulsion. In certain preferred embodiments, the carrier portion of the polyester urethane or polyether polyester urethane component may be substantially aqueous. In those and other embodiments, the polyester urethane may include a polyether polyol component. In certain preferred embodiments, the polyester urethane may be the reaction product of an aliphatic polyisocyanate and a polyester polyol. In some embodiments, the polyester urethane may comprise the reaction of an aliphatic polyisocyanate, a polyether polyol, and a polyester polyol. In yet other embodiments, the polyester urethane or polyether polyester urethane of the coating composition may comprise the reaction residue of an aromatic polyisocyanate.

The polyether component of the medical device coating composition may comprise a homopolymer, a copolymer, or a block copolymer. It may have a linear, branched or comb-like structure. In preferred embodiments, the polyether component of the medical device coating composition is only marginally soluble in water. Excessive solubility may lead to premature loss of the polyether from the surface of the coated medical device. Accordingly, it may be desirable to include one or more additional components as comonomers or to bridge successive polyether segments with, for example, urethane linked segments such as would result from a reaction between an aliphatic or aromatic polyurethane and a polyether polyol. Such reaction products typically have higher molecular weights and reduced water solubility. Polyether urethanes are particularly useful as are linear and branched polyethylene oxides and polypropylene oxides.

In some embodiments, it may be desirable add crosslinking agents to the coating composition, particularly to enhance retention of the polyether component. Such agents may be reactive toward one or both of the polyester urethane component or the polyether component. When the polyester urethane component and/or the polyether component contains active hydrogen moieties, conventional crosslinking chemistries such as polyisocyanates, polyaziridines, or other species known in the art may be employed. Alternatively, a secondary reactive species may be incorporated into one or both of the polyester urethane component or the polyether component. In a non-limiting example, pendant acrylate groups, which may be caused to undergo free radical polymerization upon exposure to UV radiation, an electron beam, and the like, may be incorporated into the polymer of the polyester urethane component and/or the polyether component.

Although not required, the coating composition may include a variety of optional components such as colorants, plasticizers, and the like so long as they do not unduly interfere with the lubricious nature of the coating. For example, colorants may be added to some or all of the coating composition to facilitate identification of a particular version of the medical device or to improve visual or radiographic contrast between the medical device and the surrounding environment.

Objects and advantages of this invention are further illustrated by the following examples, but the particular materials and amounts thereof recited in these examples, as well as other conditions and details, should not be construed to unduly limit this invention.

### EXAMPLE

List of Materials

| Trade | | |
|---|---|---|
| Designation/ | | |
| Material | Source | Description |
| Sancure 1073C | Lubrisol Advanced | 30% Solids Aliphatic Waterborne |
| | Materials, Inc., | Urethane Polymer |
| | Cleveland, OH | |
| Tecogel TPU | Lubrisol Advanced | Aliphatic Polyether-based |
| TG-500 | Materials, Inc., | Polyurethane |
| | Cleveland, OH | |

### Example 1

A 5% by weight solution of Tecogel TPU TG-500 was prepared by mixing 26.3 grams of Tecogel TG-500 with 400 grams isopropyl alcohol and 100 grams of distilled water. 70 ml of the resulting solution was combined with 30 ml of Sancure 1073C which had been diluted in a 1:4 ratio with a 20% aqueous solution of 1-methyl-2-pyrrolidinone with good mixing for 10 minutes. The resulting coating composition was applied by spraying onto a stainless steel mandrel followed by drying the resulting coating at 130° C. for 5 minutes. The coating was found to have a nominal thickness of 3-5 micron.

### Example 2

Tecogel TG-500 and Sancure 1073C solutions, prepared as in Example 1, were applied by separate spray heads to a stainless steel mandrel followed by drying the resulting coating at 130° C. for 5 minutes. The coating was found to have a nominal thickness of 3-5 micron.

The coated mandrels prepared in Examples 1 and 2 were evaluated for friction using a proprietary test and found to have friction values of 10-15 grams. A commercially available lubricious medical coating evaluated under the same conditions resulted in friction values of 14-20 grams.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

1. A medical device coating composition comprising a polyester urethane and a polyether in a substantially aqueous carrier.

2. The medical device coating composition of claim 1 wherein the polyester urethane is provided as a colloidal dispersion or an emulsion.

3. The medical device coating composition of claim 1 wherein the polyester urethane includes a polyether polyol component.

4. The medical device coating composition of claim 3 wherein the polyester urethane including a polyether polyol component is a colloidal dispersion or an emulsion.

5. The medical device coating composition of claim 1 wherein the polyester urethane is a reaction product of a mixture comprising an aliphatic polyisocyanate and a polyester polyol.

6. The medical device coating composition of claim 1 wherein the polyether is a reaction product of a mixture comprising an aliphatic polyisocyanate and a polyether polyol.

7. A method of coating a medical device, the method comprising:
providing a medical device;
providing a polyester urethane in a substantially aqueous carrier;
providing a polyether in a substantially aqueous carrier;
combining the polyester urethane in a substantially aqueous carrier and the polyether in a substantially aqueous carrier;
applying the combination of the polyester urethane and the polyether in an aqueous carrier to the medical device; and
removing the aqueous carrier from the combination of the polyester urethane and the polyether on the medical device.

8. The method of claim 7 further comprising moistening the coated medical device.

9. The method of claim 7 wherein the applying step is carried out by a coating method selected from the group consisting of spraying, dipping, brushing, or extruding.

10. The method of claim 7 wherein the polyester urethane is a polyester polyether urethane.

11. The method of claim 7 wherein the polyester urethane is provided as a dispersion.

12. The method of claim 7 wherein the polyester urethane is provided as an emulsion.

13. The method of claim 7 wherein the polyether is a polyether urethane.

14. The method of claim 7 wherein the polyether is provided as a solution.

15. The method of claim 7 wherein the polyether is provided as a dispersion.

16. The method of claim 7 wherein the polyester urethane and the polyether are combined prior to the application step.

17. The method of claim 7 wherein the polyester urethane and the polyether are combined substantially simultaneously with the application step.

18. The method of claim 7 wherein the polyester urethane and the polyether are applied sequentially before the drying step and the combining step occurs on the medical device.

19. The method of claim 7 wherein a crosslinking agent is combined with the polyester urethane and the polyether.

20. The method of claim 19 wherein the crosslinking agent reacts with at least one of the polyester urethane and the polyether.

21. A medical device produced by the process of claim 7.

22. A medical device produced by the process of claim 8.

23. The method of claim 17 wherein combining the polyester urethane in a substantially aqueous carrier and the polyether in a substantially aqueous carrier occurs as one group of one or more spray heads applies one component of the coating composition and a second group of one or more spray heads applies a second component of the coating composition.

24. The method of claim 23 wherein the first coating composition and the second coating composition are applied simultaneously.

25. The method of claim 23 wherein the first coating composition and the second coating composition are applied sequentially.

26. The method of claim 25 wherein the first coating composition and the second coating composition are applied in alternation.

27. The method of claim 23 wherein a third group of one or more spray heads applies a third component of the coating composition.
